(19) United States
(12) Patent Application Publication
(10) Pub. No.: US 2007/0049994 A1
(43) Pub. Date: Mar. 1, 2007

(54) Pace-alert

(76) Inventors: Emil DeToffol, Albany, NY (US); Wanda Zhao Russell, Fort Worth, TX (US)

Correspondence Address: Emil DeToffol, 809 Madison Ave., Albany, NY, 12208, US

(21) Appl. No.: 11/162,025

(22) Filed: Aug. 25, 2005

(51) Int. Cl. A61N 1/00

(52) U.S. Cl. 607/63

(57) ABSTRACT

A Pace-alert detects AC electrical, high frequency, and DC as well as AC magnetic fields and alarms when the field strength is beyond the safety threshold. The threshold was set up based on pacemaker and other implantable device manufacturer's specifications and left enough safety margins to give early warning before the sources actually start interfering the implanted devices.

The present invention relates to a pace-alert.

When implanted devices are installed, most doctors warn patients to stay away from electric, magnetic field (EMF), and high frequency (RF) sources. However, EMF and RF are invisible and today's life is full of electrical appliances. How to know when to avoid electrical interference is a bothersome matter for these patients. Our Pace-alert addresses this issue.

The unit is very easy to use, and does not require a technical background.

What is claimed is:

1. A pace-alert comprising: three or more channels for testing strong said fields simultaneously; first channel for testing AC electrical field; second channel for testing high frequency (RF/microwave) field; and third channel for testing DC and AC magnetic field. Other channels are possible for detecting additional phenomena.

2. A pace-alert according to claim 1, further comprising a buzzer or other device to provide audible signal for people to be aware of the danger.

3. A pace-alert according to claim 1, further comprising four light-emitting diodes (LEDs) or other indicators to indicate the status of the unit. The lit green said LED shows the battery is on. The three or more red said LEDs represent alarms for electrical (E), high frequency (RF), and magnetic (M) fields respectively, and will be on individually or jointly when one or more sources are stronger than the thresholds.

4. A pace-alert according to claim 1, further comprising other options such as meter display, LCD display, vibrator mode or flashing light for this application.
